(19) United States
(12) Patent Application Publication
(10) Pub. No.: US 2026/0139065 A1
(43) Pub. Date: May 21, 2026

(54) TREATMENT OF AIHA WITH BAFF OR BAFF RECEPTOR INHIBITORY ANTIBODIES

(71) Applicant: NOVARTIS AG, Basel (CH)

(72) Inventors: Vldadimir BEZLYAK, Baar (CH); Tomasz LAWNICZEK, Bottmingen (CH); Emerenziana MARTURANO, Basel (CH); Miona STANKOVIC, Basel-Land (CH); Patrick URBAN, Oberwil (CH)

(21) Appl. No.: 18/996,905

(22) PCT Filed: Jul. 17, 2023
(86) PCT No.: PCT/IB2023/057257

§ 371 Date: Jan. 17, 2025

Related U.S. Application Data
(60) Provisional application No. 63/368,825, filed on Jul. 19, 2022.
(60) Provisional application No. 63/384,051, filed on Nov. 16, 2022.

(51) Int. Cl. C07K 16/28; A61P 7/06

(52) U.S. Cl. CPC C07K 16/2875; A61P 7/06; C07K 2317/92

(57) ABSTRACT

The disclosure relates to a molecule that inhibits BAFF pathway, particularly an antibody or a binding fragment thereof, particularly an anti-BAFF-R antibody, e.g. ianalumab, for use in the treatment of autoimmune hemolytic anemia (AIHA), in particular, warm autoimmune hemolytic anemia (wAIHA).

### FIELD OF THE INVENTION

The invention relates to a molecule that inhibits BAFF pathway for use in the treatment of autoimmune hemolytic anemia (AIHA), in particular, warm autoimmune hemolytic anemia (wAIHA). The invention relates to a method of treating AIHA, in particular, warm autoimmune hemolytic anemia (wAIHA), comprising administering of a molecule, typically in a therapeutically effective amount, that inhibits BAFF pathway to a subject in need thereof. Preferably the molecule is an antibody or a binding fragment thereof that binds to BAFF or binds to BAFF-R, particularly ianalumab.

### SEQUENCE LISTING

The instant application contains a Sequence Listing which has been submitted electronically in ASCII format and is hereby incorporated by reference in its entirety.

### BACKGROUND

Hemolytic anemia is a form of anemia based on hemolysis, the irregular destruction of red blood cells (RBC) through autoantibodies directed against erythrocytes. The disease may be primary or secondary (drug-induced, or associated with lymphoproliferative, autoimmune, or infectious diseases, immunodeficiencies, solid tumors, or transplants).

Auto-immune hemolytic anemia (AIHA) is a heterogenic group of disorders characterized by an autoantibody-initiated destruction of RBCs. AIHAs can be classified based on the properties of the autoantibody (see Berentsen S., Seminars in Hematology 55 (2018), 141-149).

(AIHA) can be found in two basic variants: Serologically, cases are usually classified as either warm antibody-(wAIHA) or cold antibody mediated AIHA (CAIHA). The initial step of the autoimmune hemolytic process is an antigen-antibody reaction resulting in deposition of the autoantibody on the erythrocyte surface, with or without complement fixation.

In Cold Agglutinin Disease (CAD), a variant of cAIHA, the pathogenesis has been demonstrated to be entirely dependent on the classical complement pathway (Berentsen 2018). Antigen-bound IgM (cold agglutinin) on the RBC surface binds C1q and induces the classical Complement pathway. Sequential reactions lead to the formation of C3b. Upon warming to 37° C., IgM detaches from the cell, allowing agglutinated RBCs to separate. However, C3b remains bound. The C3b-coated RBCs are sequestered by the mononuclear phagocytic system, mainly in the liver. On the surviving RBCs, C3b is cleaved, leaving a high number of C3d molecules. In some cases, complement activation proceeds beyond the C₃b step with cleavage of C5, resulting in activation of the terminal pathway and intravascular hemolysis. In CAD the autoantibodies are primarily monoclonal and in more than 90% of the IgM_{K} class

wAIHA accounts for 48-70% of patients with AIHA. wAIHA is characterized by binding of primarily polyclonal immunoglobulin (mainly IgG) to RBC antigens (Rh proteins or glycophorins A-D). This binding is referred to as “warm” because it occurs at most temperatures but is maximal at 37° C. The density of these RBC antigens is usually not high enough to fix complement, but in some instances complement also becomes attached to the RBC. The opsonized RBCs are then modified (becoming spherocytes) and eventually cleared by FcγRIII or C3b receptors on macrophages/activated lymphocytes in lymphoid organs and the spleen (extravascular hemolysis) (Jäger et al 2020, Blood Rev).

Primary wAIHA is reported to be slightly less frequent compared to secondary wAIHA, with an assumed distribution of 35-50% of all wAIHA being primary (Lechner and Jäger 2010, Blood p. 1831-8). Primary wAIHA is the most frequent type in children. For primary wAIHA, the principal indication for medical therapy is symptomatic anemia. In patients with milder and partially compensated hemolytic anemia (e.g., with hemoglobin (Hb) level >10 g/dL), patients are generally monitored without treatment, i.e., “watch and wait” strategy, after weighing its potential burdens and benefits. Spontaneous remission is very uncommon, and the majority of patients require treatment as disease is usually acute and severe, however, many of the treated patients do not achieve or sustain a complete remission.

wAIHA can also be secondary to underlying diseases, or it can be caused by various medicinal products as well as organ transplantation (Kalfa 2016). In adults, the most frequent underlying disorders associated with wAIHA are lymphoproliferative malignancies, such as chronic lymphocytic leukemia (CLL), and connective tissue disorders, especially SLE. In children, the most frequently reported causes for secondary wAIHA are Evans syndrome (ES), autoimmune diseases, and infections. Management of secondary wAIHA depends on the underlying cause. While secondary wAIHA associated with autoimmune diseases like SLE or ES, is managed using a similar approach, i.e., use of steroids and immunosuppressive agents, secondary wAIHA associated with lymphoproliferative disorders, immunodeficiencies and other conditions should be treated with targeted therapy as per current guidelines according to age and the underlying disease.

To date, there are no approved therapies for the treatment of AIHA and especially wAIHA.

### BRIEF DESCRIPTION OF THE FIGURES

FIG. 1: Simulations for the ianalumab PK (top panel) and B cell count (middle panel) were obtained from the data-driven PK/PD model, and for the bottom panels from the hypothesis-driven tissue RO model. Shaded area is the 90% prediction interval (5^{th} to 95^{th} percentile), and thick line is the predicted median. Black dotted lines in the top panel represent trough concentration of 1.8 μg/mL for 300 mg q4w sc as identified in study CVAY736A2201 in patients with primary Sjögren's Syndrome. Gray dashed lines represent LLOQ at 0.025 μg/mL, B cell depletion threshold at 10 cells/μL and 90% tissue receptor occupation (RO).

FIG. 2: Auto-antibodies reduction at week 12 and week 24 from baseline (expressed as geometric mean ratio to baseline) in patients participating primary Sjögren's syndrome trial: SSAro52 (FIG. 2A), SSAro60 (FIG. 2B) and SSB autoantibodies (FIG. 2C).

FIG. 3: Auto-antibodies reduction at week 12 and week 24 from baseline (expressed as geometric mean ratio to baseline) in patients participating Systemic Lupus erythematosus (SLE) trial: C1q (FIG. 3A) and anti-dSDNA (FIG. 3B).

### DETAILED DESCRIPTION

In absence of approved therapies for AIHA, e.g. wAIHA, existing treatment options are limited. Available therapies are mostly symptomatic with limited efficacy, where responses are typically not durable/sustained after treatment discontinuation and can be associated with significant side effects as well as negative impact on quality of life. This highlights the need for new therapies of AIHA, e.g. wAIHA, that can induce durable responses or complete response, allowing the discontinuation of the treatment and the patients hemoglobin level remains within normal range or at least above 10 g/dL (treatment-free remission [TFR]).

It has now been found that inhibition of the BAFF pathway could lead to treatment of AIHA, e.g. wAIHA. Thus in one aspect, the present invention provides a molecule that inhibits BAFF pathway for use in the treatment of autoimmune hemolytic anemia, e.g. wAIHA, in a human.

The molecule is a small chemical molecule or a large biological molecule, such as an antibody or a binding fragment thereof. The molecule can also be a RNA molecule that interfere with the protein expression of BAFF pathway. Furthermore the molecule can be a peptide, e.g. a BAFF antagonist, e.g. a BAFF analogue that binds to BAFF-R but does not activate BAFF-R.

In one embodiment the molecule is a BAFF inhibitor. Normally a BAFF inhibitor binds to BAFF and prevents BAFF from binding to its receptor BAFF-R. Alternatively or additionally, a BAFF inhibitor binds to BAFF and accelerates the clearance of BAFF from the blood stream.

In one embodiment the molecule is a BAFF-R inhibitor. Normally a BAFF-R inhibitor binds to BAFF-R and prevents BAFF-R from binding to BAFF. Alternatively a BAFF-R inhibitor binds to a BAFF-R, while not preventing BAFF-R from binding to BAFF, changes the conformation of BAFF-R and thereby preventing or reducing the BAFF-R activation triggered by BAFF binding (allosteric inhibition).

In one embodiment the molecule is an antibody or a binding fragment thereof.

In one embodiment the antibody or a binding fragment thereof is a BAFF inhibitor, which is also called an anti-BAFF antibody or a binding fragment thereof. In one embodiment the anti-BAFF antibody or a binding fragment thereof is belimumab or a binding fragment thereof. In one embodiment the anti-BAFF antibody or a binding fragment thereof is tibulizumab or a binding fragment thereof.

In one embodiment, the antibody or a binding fragment thereof comprises CDR-H1, CDR-H2, and CDR-H3 having the amino acid sequences of SEQ ID NO:11, SEQ ID NO: 12, and SEQ ID NO: 13, and CDR-L1, CDR-L2, and CDR-L3 having the amino acid sequences of SEQ ID NO: 14, SEQ ID NO: 15, and SEQ ID NO: 16, respectively.

In one embodiment, the antibody or a binding fragment thereof comprises a heavy chain having the amino acid sequences of SEQ ID NO:9 and a light chain having the amino acid sequences of SEQ ID NO:10, respectively.

In one embodiment the antibody or a binding fragment thereof is a BAFF-R inhibitor, which is also called an anti-BAFF-R antibody or a binding fragment thereof. In one embodiment the anti-BAFF-R antibody or a binding fragment thereof is ianalumab or a binding fragment thereof.

B-cells play an important role in the pathogenesis of autoimmune diseases. B-cell depletion with the anti-CD20 monoclonal antibody rituximab is an established approach to treat autoimmune diseases, including wAIHA. However, the use of rituximab is associated with some limitations, including insufficient B-cell depletion, especially in tissues, and persistence of rituximab-resistant B-cells that play a critical role in autoimmune disease refractoriness and chronicity (Mahévas et al 2015, J Autoimmun p. 22-30, Crickx et al 2021, Sci Transl Med.).

Recent studies have demonstrated that the BAFF-R signaling pathway is involved in the activation of B-cell effector functions such as antibody production, isotype class switching, B-cell proliferation, maturation, and survival (Mackay and Schneider 2009, Nat Rev Immunol p. 491-502). BAFF overexpression results in disruption of B-cell immune tolerance and consequently autoimmune disorders (Mackay and Browning 2002, Nat Rev Immunol p. 465-75). Increased concentrations of soluble BAFF (B-cell Activating Factor) were found in various autoimmune diseases, including Sjögren syndrome (SS) and pemphigus vulgaris (PV). Increased serum BAFF levels were detected in wAIHA patients with low hemoglobin (<8 g/dL) and high lactate dehydrogenase activity (Xu et al 2015, Int J Hematol p. 394-400).

Targeting the BAFF pathway in autoimmune diseases such as Systemic Lupus erythematosus (SLE) and Lupus nephritis (LN) to improve clinical outcomes has been successfully demonstrated with the monoclonal antibody belimumab (BENLYSTA® US Prescribing Information). A recent clinical study (Mahevas et al., Haematologica 2020 p. 2449-2457) tested the anti-BAFF antibody belimumab (5 doses of 10 mg/kg i.v. for up to 12 weeks) given together with rituximab (2 doses of 1000 mg i.v. for up to 2 weeks) in 15 patients with persistent or refractory ITP, an autoimmune disease pathophysiologically related to wAIHA. Eighty percent of patients responded to this combination and maintained the response for 12 months.

It also has been shown that enhanced antibody-dependent cellular cytotoxicity (ADCC), as a result of using afucosylated antibodies, can improve clinical outcomes in autoimmune diseases such as lupus nephritis (LN) (Furie et al 2021, Ann Rheum Dis).

Ianalumab is a glycoengineered (afucosylated), fully human IgG1 monoclonal antibody directed against BAFF-R, see e.g. WHO Drug Information, Vol. 34, No. 2, page 426-7, 2020, “proposed INN: List 123, under “p. 280-281 ianalumabum”. This antibody may be expressed, preferably in a host cell which lacks fucosyl-transferase, for example in a mammalian cell line with an inactive FUT8 gene (e.g. FUT8^{-/-}), to provide a functional non-fucosylated antibody.

Ianalumab is expressed on the surface of B-cells, thereby targeting B-cells and their function with two main modes of action:
1. Direct lysis and depletion of BAFF-R-expressing B-cells by enhanced antibody-dependent cellular cytotoxicity (ADCC) due to its fucose-deficient Fc region; and
2. BAFF receptor blockade that interrupts BAFF-mediated signaling for B-cell activation, maturation, proliferation, and survival.

In preclinical studies, ianalumab significantly enhanced ADCC against B-cells and cytokine production by NK cells, and was more potent in depleting B-cells when compared to approved anti-CD20 antibodies, including rituximab, obinutuzumab and ofatumumab (McWilliams et al 2019).

The antigen specificity of ianalumab is directed against the BAFF receptor, BAFF-R (Syn: BR3), which is expressed on the surface of immature and mature B cells up to the lymphoblast stage. Pro- and pre-B cells that express CD20 but not BAFF-R are therefore not expected to be affected by ianalumab. Hence, compared to the existing B cell depleting anti-CD20 antibody therapies, ianalumab targets a narrower spectrum of more differentiated B cells.

So far, ianalumab has been investigated across multiple indications in autoimmune disorders and hematologic malignancies. Data from the ongoing and completed studies shows that treatment with ianalumab was generally safe and well tolerated. Among others, ianalumab demonstrated efficacy in primary Sjögren's syndrome (pSS) in a phase II study (Bowman S J, et al (2021)).

Through its dual mechanism of action, ianalumab is expected to be able to overcome the above-described limitations of currently available B-cell depleting therapy such as rituximab, by potent B-cell depletion (ADCC) in combination with BAFF-R blockade, thereby preventing the BAFF-driven pathogenic rebound and resistance mechanisms, and thus provides a basis for treatment of AIHA, especially wAIHA. More generally, promising treatment with a dual action approach targeting 1) the BAFF-receptor pathway as well as 2) B-cell depletion can address the unmet medical needs in AIHA, especially wAIHA, also in cases of CD20-negative B cells.

Hence the development of BAFF inhibiting molecules, such as anti-BAFF and anti-BAFF-R antibodies (anti-BAFF-Receptor antibodies), especially ianalumab, in hematological autoimmune diseases, including AIHA and especially wAIHA, is supported by one, more than one or all of the following:
B-cell depletion is an established therapeutic measure in the treatment of AIHA, e.g. wAIHA;
BAFF-R pathway inhibition has been demonstrated to be effective and safe in nonhematologic autoimmune diseases (BENLYSTA® in SLE and LN);
BAFF blockade combined with B-cell depletion has been demonstrated to be effective and safe in ITP, a condition belonging to the same family of hematological autoimmune diseases as wAIHA (Mahévas et al 2021);
Clinical studies with ianalumab have demonstrated its efficacy in other autoimmune diseases (e.g., RA, pSS);
Ianalumab demonstrated a favorable benefit-risk profile based on data from 480 patients exposed to ianalumab in several autoimmune diseases.

The use of anti-BAFF-R antibodies in the case of AIHA and especially wAIHA allows for a short treatment term, especially when compared to the treatment terms used in treatment of Sjögren's Disease or the like. In addition, while with other anti B-cell treatments known so far, e.g. with rituximab, resistance develops (which appears to be related at least partially to an overcompensating biosynthesis of BAFF), the dual mechanism of action of anti-BAFF-R antibodies should provide a barrier to such resistance development. Such a resistance will likely not be found with the BAFF-R blocking antibodies used in the present treatment. In addition, an advantage of inhibition of BAFF-R and not BAFF ligand may result from the potential that the interaction of BAFF with other possible binding sites for BAFF apart from BAFF-R, like TNFRSF13B/TACI, TNFRSF13, TNFRSF13C or TNFRSF17/BCMA, is likely less or not affected, so that side effects based on inhibition of such other binding sites are diminished or abolished.

In one embodiment, the invention relates to an anti-BAFF antibody, e.g. belimumab, or an anti-BAFF-R antibody, e.g. ianalumab, or a binding fragment thereof, respectively, especially ianalumab, for use in the treatment of primary wAIHA or of secondary wAIHA. In one embodiment, the secondary wAIHA is not due to lymphoproliferative diseases (e.g. CLL), immunodeficiencies or other immunologic diseases requiring targeted treatment, or immunosuppressant therapies.

The pathogenesis and clinical manifestation of primary and several secondary wAIHA are similar and associated with disruption of B-cell immune tolerance. Therefore, these patients are expected to benefit from present invention.

In one embodiment, the invention relates to an antibody or a binding fragment thereof for use in the treatment of AIHA, e.g. wAIHA, wherein the antibody or binding fragment thereof comprises CDR-H1, CDR-H2, and CDR-H3 having the amino acid sequences of SEQ ID NO: 3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

In one embodiment, the invention relates to an antibody or a binding fragment thereof for use in the treatment of AIHA, e.g. wAIHA, wherein the antibody or a binding fragment thereof comprises VH having the amino acid sequences of SEQ ID NO: 17, and VL having the amino acid sequences of SEQ ID NO: 18, respectively.

In one embodiment, the invention relates to an antibody or a binding fragment thereof for use in the treatment of AIHA, e.g. wAIHA, wherein the antibody or a binding fragment thereof comprises a heavy chain having the amino acid sequence of SEQ ID NO: 1 and a light chain variable region having the amino acid sequence of SEQ ID NO: 2.

The doses, regimen, route of administration, and treatment duration were chosen with the aim to 1) achieve rapid, profound and consistent depletion of B cells, 2) complete inhibition of the BAFF-R pathway in both blood and tissues, and 3) with a limited treatment duration, to induce quick and durable responses that are maintained after the end of the treatment period, allowing patients to stay off treatment with adequate hemoglobin levels.

Thus, in one embodiment, the invention relates to an anti-BAFF antibody, e.g. belimumab, or an anti-BAFF-R antibody, e.g. ianalumab, or a binding fragment thereof, respectively, especially ianalumab, wherein the antibody or a binding fragment thereof is to be administered at a dose in the range of from about 1 to 30 mg/kg, in particular from about 1 to 27 mg/kg, more particularly in the range from about 1 to 10 mg/kg.

In one embodiment, the invention relates to an anti-BAFF antibody, e.g. belimumab, or an anti-BAFF-R antibody, e.g. ianalumab, or a binding fragment thereof, respectively, especially ianalumab, is to be administered to a subject in need thereof once every 2, 3, 4, 6 or 8 weeks, especially every 4 weeks (+/−3 days).

In one embodiment the administration is via intravenous route or subcutaneous route, particularly via intravenous route.

In one embodiment, the invention relates to the anti-BAFF-R antibody or a binding fragment thereof for use in the treatment of AIHA, e.g. wAIHA, wherein the anti-BAFF-R antibody or binding fragment thereof, e.g. ianalumab, is to be administered at a dose of about 1 to 10 mg/kg, about 3 mg/kg to 9 mg/kg, about 3 mg/kg or about 9 mg/kg. In one embodiment the anti-BAFF-R antibody or a binding fragment thereof, e.g. ianalumab, is to be administered to a subject in need thereof once every 4 weeks (+/−3 days). In one embodiment, ianalumab is to be administered at a dose of 3 mg/kg, once every four weeks (+/−3 days). In one embodiment, ianalumab is to be administered at a dose of 9 mg/kg, once every four weeks (+/−3 days). In one embodiment the administration is via intravenous route or subcutaneous route, particularly via intravenous route.

In one embodiment, the invention relates to an anti-BAFF antibody, e.g. belimumab, or an anti-BAFF-R antibody, e.g. ianalumab, or a binding fragment thereof, respectively, especially ianalumab, wherein the antibody or a binding fragment thereof is to be administered not more than 12 doses, not more than 9 doses, not more than 6 doses, not more than 4 doses. In one embodiment the antibody or binding fragment thereof is to be administered at least 3 doses or at least 4 doses. In one embodiment, the antibody or binding fragment thereof is to be administered between 4 to 9 doses, 4 to 8 doses, or 4 to 6 doses.

In one embodiment, the anti-BAFF-R antibody or a binding fragment thereof, e.g. ianalumab, for use in the treatment of AIHA, e.g. wAIHA, wherein the antibody or a binding fragment thereof is to be administered in total of 8 doses, 6 doses, or 4 doses, in particular in a total of 4 doses. In one embodiment, wherein the doses are administered consecutively, normally every four weeks (monthly).

In one embodiment, the invention relates to the anti-BAFF-R antibody or a binding fragment thereof, e.g. ianalumab, for use in the treatment of AIHA, e.g. wAIHA, wherein the anti-BAFF-R antibody or a binding fragment thereof is to be administered during a period of up to, about 12 months, about 9 months, about 6 months or about 5 months, after the first dose administration on day 1, respectively; or wherein the anti-BAFF-R antibody or a binding fragment thereof is to be administered during a period of up to about 4 months, preferably about 3 months, after the first dose administration on day 1, respectively; or wherein the anti-BAFF-R antibody or a binding fragment thereof is to be administered during a period of up to 20 weeks (+/−3 days), after the first dose administration on day 1, respectively; or wherein the anti-BAFF-R antibody or a binding fragment thereof is to be administered during a period of up to 16 weeks (+/−3 days), after the first dose administration on day 1; or wherein the anti-BAFF-R antibody or a binding fragment thereof is to be administered during a period of up to 12 weeks (+/−3 days), after the first dose administration on day 1.

In one embodiment, the invention relates to ianalumab for use in the treatment of AIHA, e.g. wAIAH, or relates to a method of treating AIHA, e.g. wAIHA, comprising administering ianalumab, typically in a therapeutically effective amount, in a subject in need thereof, wherein the subject achieves a complete response (CR), normally after the 2nd or more likely after the 3rd or after the 4 dose. CR is judged by any one, any two or all of the below effects.:
(1) normalization of hemoglobin (Hb level ≥about 11 g/dL (women) or ≥12 about g/dL (men);
(2) no evidence of hemolysis (normal levels of indirect bilirubin, LDH, haptoglobin, and/or reticulocytes);
(3) absence of red blood cells transfusions.

In one embodiment the complete response refers the normalization of the hemoglobin (Hb level ≥11 g/dL (women) or ≥12 g/dL (men)). In one embodiment, CR is judged by achieving all the above effects.

In one embodiment, the invention relates to ianalumab for use in the treatment of AIHA, e.g. wAIAH, or relates to a method of treating AIHA, e.g. wAIHA, comprising administering ianalumab, typically in a therapeutically effective amount, in a subject in need thereof, wherein the subject achieves a durable response, normally after the 2nd or more likely after the 3rd dose or more likely after the 4^{th} dose, preferably monthly dose, preferably at a dose range of 3 mg/Kg to 9 mg/Kg. The term “durable response” as used here, is judged by either one, or both of the below effects (binary effects) and the effect(s) lasts at least 3, 4, 5, 6, 7 consecutive weeks:
(1) the hemoglobin blood concentration of the subject has reached 10 g/dL;
(2) the hemoglobin blood concentration of the subject has increased by about 2 g/dL or more than baseline

In one preferred embodiment, the effect(s) last for at least 8 consecutive weeks. In one further preferred embodiment, durable response with binary effects lasts for at least 8 consecutive weeks.

In one embodiment, the invention relates to ianalumab for use in the treatment of AIHA, e.g. wAIAH, or relates to a method of treating AIHA, e.g. wAIHA, comprising administering ianalumab, typically in a therapeutically effective amount, in a subject in need thereof, wherein the hemoglobin blood concentration of
(1) a male subject has reached about 10 g/dL or more, about 11 g/dL or more, and preferably about 12 g/dL or more; or
(2) a female subject is maintained about 9 g/dL or more, about 10 g/dL or more, and preferably about 11 g/dL s maintained about 10 g/dL or more, about 11 g/dL or more, on or after the treatment, normally after the 2nd or more likely after the 3rd or after the 4^{th} dose.

In one embodiment, the invention relates to ianalumab for use in the treatment of AIHA, e.g. wAIAH, or relates to a method of treating AIHA, e.g. wAIHA, comprising administering aianalumab, typically in a therapeutically effective amount, in a subject in need thereof, wherein the subject does need blood transfusion on and after the treatment, normally after the 2nd or more likely after the 3^{rd} or 4^{th} dose. In one embodiment, the subject does not need blood transfusion for at least 4 months, at least 6 months, at least 9 months, at least one year, at least 18 months, at least two years or at least three years, starting from day 1 of the treatment.

In one embodiment, the invention relates to ianalumab for use in the treatment of AIHA, e.g. wAIAH, or relates to a method of treating AIHA, e.g. wAIHA, comprising administering ianalumab, typically in a therapeutically effective amount, in a subject in need thereof, wherein the subject does not have evidence of hemolysis on and after the treatment, normally after the 2nd or more likely after the 3^{rd} or 4^{th} dose. Evidence of hemolysis includes abnormality of one, two, three or all of the biomarkers selected from a group consisting of bilirubin, LDH, haptoglobin, and reticulocytes. In one embodiment, the subject does not have evidence of hemolysis for at least 4 months, at least 6 months, at least 9 months, at least one year, at least 18 months, at least two years or at least three years, starting from day 1 of the treatment.

In one embodiment, the invention relates to ianalumab for use in the treatment of AIHA, e.g. wAIAH, or relates to a method of treating AIHA, e.g. wAIHA, comprising administering a ianalumab, typically in a therapeutically effective amount, in a subject in need thereof, wherein the hemoglobin blood concentration of the subject in need thereof has increased by about 1 g/dL or more, especially about 2 g/dL or more, especially about 3 g/dL or more, especially about 4 g/dL or more, than baseline after administration of ianalumab, on or after the treatment, normally after the 2nd or more likely after the 3^{rd} or 4^{th} dose.

Further to any one of the preceding embodiments, the hemoglobin blood concentration of the subject is measured at the time of the 3^{rd} dose administration, either right before or after the 3^{rd} administration, normally on the same day. In one embodiment, the hemoglobin blood concentration of the subject is measured between week 9 to week 25 after the first administration, especially when the drug is dosed once every four weeks.

In one embodiment, the hemoglobin blood concentration of the subject is measured weekly, bi-weekly or monthly. In one embodiment, the hemoglobin blood concentration of the subject is measured biweekly till week 25 and monthly thereafter, taking the first administration as week 1, day 1.

In one embodiment, the use of the invention according to any one of the preceding embodiments, especially with ianalumab, wherein the treatment discontinues after 8 doses, after 6 doses, or preferably after 4 doses. Normally those doses are administered consecutively. Normally the drug is administered every four weeks.

In one embodiment, the invention relates to ianalumab for use in the treatment of AIHA, e.g. wAIAH or relates to a method of treating AIHA, e.g. wAIHA, comprising administering ianalumab, typically in a therapeutically effective amount, in a subject in need thereof, where the treatment discontinues when the subject achieves a complete response, normally and typically, when the complete response last for at least three weeks, normally for three weeks or one month, during which at least 2 measures are performed.

In one embodiment, the invention relates to ianalumab for use in the treatment of AIHA, e.g. wAIAH or relates to a method of treating AIHA, e.g. wAIHA, comprising administering ianalumab, typically in a therapeutically effective amount, in a subject in need thereof, where the treatment discontinues when the hemoglobin blood concentration of the subject reaches 10 g/dL or more and lasts, normally and typically, for at least three weeks, normally for three weeks or one month, during which at least 2 measures are performed.

In one further embodiment, the treatment discontinues when the hemoglobin blood concentration of the subject
(1) is maintained about 10 g/dL or more for 3 consecutive weeks; and
(2) has ≥2 g/dL increase from baseline for 3 consecutive weeks.

The hemoglobin blood concentration of the subject is normally measured between week 9 to week 25 after the first administration, especially when the drug is dosed once every four weeks.

In one embodiment, the invention relates to ianalumab for use in the treatment of AIHA, e.g. wAIAH or relates to a method of treating AIHA, e.g. wAIHA, comprising administering ianalumab, typically in a therapeutically effective amount, in a subject in need thereof, wherein the duration of response is at least about 3 months, at least about 6 months, at least about 9 months, or at least about 12 months. In one further embodiment, the duration of response is about 12 months, about 15 months, about 18 months, or about 24 months.

The term “duration of response”, as used herein, especially in the context of a clinical trial settings, e.g. example 2, refers to subjects who previously reached durable response (binary effect for at least 8 consecutive weeks): the time from the first hemoglobin assessment showing durable response to confirmed loss of durable response, defined as the first of the following events:
hemoglobin level below 10 g/dL in at least two consecutive weekly assessments* (date of the first of the 2 relapsed assessments will be considered as relapse date),
start of any rescue or prohibited treatment,
death (whatever the cause).

The following treatments are considered as rescue medications:

any additional wAIHA-directed treatment which is not listed among prohibited medication
an increase in the dosage of the supportive care >20% comparing to the pre-enrollment dose, which is in line with the recommendations with opinions of the experts. Re-start of allowed supportive treatments at the pre-enrollment dose or return to the pre-enrollment dose level after dose decrease are not considered as a rescue medication.
from Week 8 Day 1 onwards, transfusions is considered a rescue treatment as it could influence the Hb level and confound the efficacy of ianalumab.

Prohibited medications are live or live attenuated vaccines, i.v. or oral immunosuppressive drugs, rituximab, belumimab or other monoclonal antibodies, and other experimental therapies.

In one embodiment, the invention relates to ianalumab for use in the treatment of AIHA, e.g. wAIAH or relates to a method of treating AIHA, e.g. wAIHA, comprising administering ianalumab, typically in a therapeutically effective amount, in a subject in need thereof, wherein the subject enters the stage of treatment free remission (TFR). The term “treatment free remission (TFR)”, as used herein, refers the period of time, during which the subject staying off of treatment with temporary or permanent decrease or subsidence of manifestations of a disease. In TFR, the subject normally and typically maintains hemoglobin blood concentration about 10 g/dL or more, typically and preferably, about 11 g/dL or more for women and about 12 g/dL or more for men. In TFR, the subject normally does not receive transfusion. In TFR, the treating physician normally does not consider the subject needs any treatment.

In one embodiment, the subject has treatment free remission (TFR) for at least about 3 months, for at least about 6 months, for at least about 9 months, for at least about 12 months or for up to about 2 years, up to about 3 years, up to about 4 years.

The term “relapse”, as used herein, refers to a drop of hemoglobin below the normal level (e.g. about 12 g/dL and above for a man and about 11 g/dL and above for a woman) and normally stay below the normal level for a period of time. In most of the cases, the term “relapse” refers to a drop of hemoglobin below about 10 g/dL. The period of time is normally for at least 2 consecutive measures, normally the consecutive measures are performed less than 2 months apart, less than one month apart, less than 3 weeks apart, less 2 weeks apart, one week apart.

In one embodiment, the invention relates to ianalumab for use in the treatment of AIHA, e.g. wAIAH or relates to a method of treating AIHA, e.g. wAIHA, comprising administering ianalumab, typically in a therapeutically effective amount, in a subject in need thereof, wherein the subject has relapsed after TFR. The TFR can be achieved through the administration of (1) an anti-BAFF antibody, e.g. belimumab; (2) an anti-BAFF-R antibody, e.g. ianalumab, or a binding fragment thereof; or (3) neither of the above but another wAIHA treatment. In one embodiment, the TFR is achieved through the administration of ianalumab.

In one embodiment, the invention relates to ianalumab for use in the treatment of AIHA, e.g. wAIAH or relates to a method of treating AIHA, e.g. wAIHA, comprising administering ianalumab, typically in a therapeutically effective amount, in a subject in need thereof, wherein the subject has relapsed after TFR, the TFR is achieved through the administration of ianalumab (re-treatment). In one further embodiment, the subject receives the same dosing regimen as he received before achieving TFR. a subject naive of ianalumab treatment. In one embodiment, the subject receives less doses than the doses he had received before he achieved TFR. In one embodiment, the subject receives more doses than the doses he had received before he achieved TFR. The term “dose” as used in these re-treatment embodiments, refers to the amount in a single administration, the dosing interval, the total treatment duration or any one, two or all of the combination of the above.

In one embodiment, the invention relates to the anti-BAFF-R antibody or a binding fragment thereof, e.g. ianalumab, for use according to any one of the preceding embodiments, wherein the anti-BAFF-R antibody or binding fragment thereof is or is to be administered as monotherapy for AIHA, e.g. wAIHA.

In one embodiment, the invention relates to an anti-BAFF antibody, e.g. belimumab, or an anti-BAFF-R antibody, e.g. ianalumab, or a binding fragment thereof, respectively, especially ianalumab, for use in the treatment of warm autoimmune hemolytic anemia (wAIHA) in a human, wherein the subject failed at least one previous line of treatment. The previous treatment can be the same or a different wAIHA treatment.

Different wAIHA treatment includes but not limited to treatment with a corticosteroid, e.g. prednisolone, prednisone or equivalent thereof, treatment with a B-cell depleting agent, such as rituximab. In one embodiment, the anti-BAFF-R antibody or a binding fragment thereof, e.g. ianalumab, is administered while tapering out a corticosteroid. In one embodiment, the subject did not receive a B-cell depleting agent, such as rituximab, at least 12 weeks prior to administration according to the present invention. In an alternative embodiment, the subject received a B-cell depleting agent, such as rituximab, prior to being treated with the present invention, especially prior to being treated with ianalumab. Normally the wash out period is at least 12 weeks prior to administration according to the present invention.

In one embodiment, the invention relates to the anti-BAFF-R antibody or a binding fragment thereof for use according to any one of the preceding embodiments, wherein the anti-BAFF-R antibody or binding fragment thereof is to be administered in combination with one or more additional agents. The one or more agents includes but not limited to corticosteroid, especially prednisone or prednisolone or equivalent thereof, especially in a low dose regimen (e.g. 15 mg/day or less, e.g. 1 to 10, such as 2 to 5 mg/day prednis(ol)one) or while tapering the corticosteroid out; danazole (e.g. 200 mg three times daily; and erythropoietin (e.g. erythropoietin alpha, beta or delta 10 00 units/week).

In one embodiment, the invention relates to an anti-BAFF antibody, e.g. belimumab, or an anti-BAFF antibody, e.g. ianalumab, or a binding fragment thereof, respectively, especially ianalumab, wherein the antibody or a binding fragment thereof is to be administered to a subject, wherein the subject has primary or secondary wAIHA and the subject had an insufficient response to, or relapsed after at least one line of treatment, including patients with steroid resistance, dependence or intolerance. Typically the subject has wAIHA that has been previously documented by positive direct antiglobulin test (DAT) specific for anti-IgG or anti-IgA.

Steroid resistance: failure to obtain hematologic response within 3 weeks on at least 1 mg/kg predniso(lo)ne. Steroid dependence: Need to continue predniso(lo)ne at a dose of >10 mg/day to maintain a response. Steroid intolerance: side effects or anaphylactic/anaphylactoid reactions which lead to permanent termination of steroids.

In one embodiment, the invention relates to an anti-BAFF antibody, e.g. belimumab, or an anti-BAFF antibody, e.g. ianalumab, or a binding fragment thereof, respectively, especially ianalumab, for use in the treatment of wAIHA, wherein the subject has a hemoglobin blood concentration of 10 g/mL or less, 9 g/dL or less than 9 g/dL, 8 g/dL or less than 8 g/dL, 7 g/dL or less than 7 g/dL, 6 g/dL or less than 6 g/dL, 5 g/dL or less than 5 g/dL. In one embodiment the subject has a hemoglobin blood concentration of 10 g/mL or less. Typically the subject has associated with presence of symptoms related to anemia.

In one embodiment, Neutrophils of the subject is more than 1000/mm³

In one embodiment, the Serum creatinine in the subject is normal or is lower than 1.5×upper limit of normal (ULN).

In one embodiment, the subject is an adolescences 12 to 17 years old. The toxicology studies with ianalumab in Cynomolgus monkey were conducted in the following age range: 2.2 to 3.5 years (preliminary single/multiple dose study); 5 to 6 years (4-week study); 4 to 5 years (13-week study); 4 to 6 years (26-week study). No safety issues relevant to humans were identified in these studies. The age translation follows the general concept that 1 primate year is considered equivalent to 4 human years (Baldrick 2010). This covers the human age range of 8.8-24 years, thereby covering the age of adolescent patients planned to be included in the proposed phase 3 trial.

In one aspect, the invention relates to a method of treating a subject suffering from autoimmune hemolytic anemia (AIHA), e.g. warm autoimmune hemolytic anemia (wAIHA), wherein the method comprises administering an anti-BAFF antibody, e.g. belimumab, or an anti-BAFF antibody, e.g. ianalumab, or a binding fragment thereof, respectively, especially ianalumab, typically in a therapeutically effective amount, to the subject in need thereof. The method in particular comprises any one of the preceding embodiments.

In one aspect, the invention relates to the use of an anti-BAFF antibody, e.g. belimumab, or an anti-BAFF antibody, e.g. ianalumab, or a binding fragment thereof, respectively, especially ianalumab, in the manufacture of a medicament for treating a subject suffering from autoimmune hemolytic anemia (AIHA), e.g. warm autoimmune hemolytic anemia (wAIHA). The use in particular comprises any one of the preceding embodiments.

### Definitions

As used herein, the following terms are intended to have the following meanings:

A, An, The: As used herein, the term “a”, “an”, “the” and similar terms used in the context of the present disclosure (especially in the context of the claims) are to be construed to cover both the singular and plural unless otherwise indicated herein or clearly contradicted by the context. As such, the terms “a” (or “an”), “one or more”, and “at least one” can be used interchangeably herein. They can stand for “one or more”, in particular, where an anti-BAFF-R antibody or binding fragment thereof is being referred to, for “one”, referring to anti-BAFF-R antibodies or binding fragments thereof of the same amino acid sequence.

“About” is in particular intended to include a deviation of the specific numerical value following this word±20%, e.g. ±10%, e.g. ±5%, or ±2%, and includes the mentioned numerical value itself.

And/or: The term “and/or” means that each one or both or all of the components or features of a list are possible variants, especially two or more thereof in an alternative or cumulative way, including any subcombination of two alternatives. It can be replaced with “one or more or all of”.

Additional Agent: For convenience, an agent that is used in combination with an anti-BAFF-R antibody, or an anti-BAFF antibody, or a binding fragment thereof, is referred to herein as an “additional” agent.

Antibody: The term “antibody” refers to a protein, e.g., an immunoglobulin chain or fragment thereof, comprising at least one immunoglobulin variable domain sequence. The term “antibody” includes, for example, a monoclonal antibody (including a full length antibody which has an immunoglobulin Fc region). An antibody comprises a full length antibody, or a full length immunoglobulin chain, or an antigen binding or functional fragment of a full length antibody, or a full length immunoglobulin chain. An antibody can also be a multi-specific antibody, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope.

Anti-BAFF-R antibody and Anti-BAFF antibody, or a binding fragment thereof: The term “anti-BAFF-R antibody”, as used herein, refers to an antibody that binds to BAFF-R. The term “anti-BAFF antibody”, as used herein, refers to an antibody that binds to BAFF. The term “binding fragment” as used herein refers to a portion of an antibody capable of binding a BAFF-R epitope, especially an epitope involved in the binding of BAFF to BAFF-R and/or negatively affecting the activation of BAFF-R by BAFF. The binding of the antibody (or a binding fragment thereof) to BAFF-R or to BAFF inhibits the binding of BAFF-R to BAFF and thereby reduces the formation of BAFF/BAFF-R complexes, and/or reduces the activation of BAFF-R. Suitably, the antibody or a binding fragment thereof may reduce the formation of BAFF/BAFF-R complexes and/or reduce the activation of BAFF-R by at least 30%, at least 40%, at least 50%, at least 60%, at least 70%, at least 80%, at least 90%, at least 95%, at least 96%, at least 97%, at least 98%, at least 99%, or more as compared to a suitable control (for example a sample without the presence of the antibody or a binding fragment thereof, e.g. in an ELISA). Additionally or alternatively, the antibody or a binding fragment thereof may dissociate preformed BAFF/BAFF-R complexes. In a suitable embodiment antibody or a binding fragment thereof may dissociate at least 30%, at least 40%, at least 50%, at least 60%, at least 70%, at least 80%, at least 90%, at least 95%, at least 96%, at least 97%, at least 98%, at least 99%, or more of preformed BAFF/BAFF-R complexes. As before, this property may be compared to a suitable control (for example a sample without the presence of the antibody or binding fragment thereof). A particular embodiment relates to an antibody or a binding fragment thereof comprising the following complementarity determining sequences (CDR): CDR-H1 (SEQ ID NO: 3), CDR-H2 (SEQ ID NO:4), CDR-H3 (SEQ ID NO:5), CDR-L1 (SEQ ID NO:6), CDR-L2 (SEQ ID NO:7) and CDR-L3 (SEQ ID NO:8). Particular embodiments of the invention comprise ianalumab or a binding fragment thereof as anti-BAFF-R antibody or a binding fragment thereof.

BAFF-R: The term “BAFF-R” refers to the B-cell activating factor receptor protein. BAFF-R is also known as TNF Receptor Superfamily Member 13C (TNFRSF13C). The human and murine amino acid and nucleic acid sequences can be found in a public database, such as GenBank, UniProt and Swiss-Prot. For example, an amino acid sequence of human BAFF-R can be found as UniProt/Swiss-Prot Accession No. Q96RJ3 and a nucleotide sequences encoding human BAFF-R can be found at Accession Nos. NM_052945.4. It is expressed predominantly on B-lymphocytes and on a subset of T-cells.

Combination: The terms “a combination” or “in combination with” is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together or separately in different compositions. In general, it is expected that additional therapeutic agents utilized in combination be utilized at levels that do not exceed the levels at which they are utilized individually. In some embodiments, the levels utilized in combination will be lower than those utilized individually.

Effective amount: By the term “effective amount” or “therapeutically effective amount” or “pharmaceutically effective amount”, is meant the amount or quantity of active agent that is sufficient to elicit the required or desired response, or in other words, the amount that is sufficient to elicit an appreciable biological response when administered to a subject. Said amount preferably relates to an amount that is therapeutically or in a broader sense also prophylactically effective against the progression of a disease or disorder as disclosed herein.

Patient/subject: As used herein, the term “patient” or “subject” are taken to mean a human. Except when noted, the terms “patient” or “subject” are used herein interchangeably.

In need of: As used herein, a subject is “in need of” a treatment if such subject would benefit biologically, medically or in quality of life from such treatment.

Treat, treating, treatment: As used herein, the term “treat”, “treating” or “treatment” (which can be exchanged with each other) of any disease or disorder refers in one embodiment to ameliorating the disease or disorder (e.g., slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition. The benefit to a patient to be treated is either statistically significant or at least perceptible to the patient or to the physician.

An anti-BAFF-R antibody (in particular, ianalumab) or binding fragment thereof and an additional agent can be administered simultaneously, in the same or in separate compositions, or sequentially. For sequential administration, the anti-BAFF-R antibody or binding fragment thereof can be administered first, and the additional agent can be administered second, or the order of administration can be reversed.

In one aspect, the invention provides a product for the separate, sequential or simultaneous or concurrent use of combination partners of the invention, especially a kit comprising two or more separate pharmaceutical compositions, at least one of which contains an anti-BAFF-R antibody, in particular ianalumab, or a binding fragment thereof, respectively, and at least one other of which comprises an additional agent. In one embodiment, the kit comprises means for separately retaining said compositions, such as a container, divided bottle, or divided foil packet. An example of such a kit is a blister pack, as typically used for the packaging of tablets, capsules and the like.

### EXAMPLES

The following example illustrates the invention without limiting the scope thereof.

### Example 1 Pharmacokinetics-Pharmacodynamic Modelling

A population PK/PD model was built using a sequential PK/PD (Zhang, J Pharmacokinet Pharmacodyn; 30 (6): 387-404. 2003) approach to describe ianalumab concentrations and CD19+B-cell count of patients from various auto-immune diseases.

### Population PK/PD Model

A two-compartment population PK model with linear clearance was fitted to i.v. and s.c. data derived from studies in RA patients and in pSS patients. The PK/PD model, was parameterized in terms of B-cell counts at baseline (Base), death rate of B cells (kout), volume of peripheral compartment for B cells (Vb), intercompartment transition rate of B cells (Qb), maximum stimulating effect of ianalumab on the death rate of B cells (Emax) and the ianalumab concentration for which VAY736 effect is half-maximal (EC50).

### PK-Tissue Receptor Occupancy (RO) Structural Model

As circulating B-cell dynamics as described by the population PK/PD model are not necessarily expected to be predictive of clinical efficacy, a hypothesis-driven tissue receptor occupancy (RO) model was also developed and used to consider the competitive binding between ianalumab and soluble BAFF (sBAFF) on BAFF-R under a quasi-steady state assumption. The following equation links the PK (C_{ianalumab}(t), nM) to the BAFF-R engagement by ianalumab (ROVAY(t), %):

$${RO}_{ianalumab} ( t ) = \frac{{C}_{ianalumab} ( t ) / {Kd}_{i a n a l u m a b}}{1 + {C}_{i a n a l umab} ( t ) / K {d}_{ianalumab} + {C}_{sBAFF} / K {d}_{s B A F F}} \times 100$$

where C_{sBAFF} is the soluble BAFF concentration in tissues (nM), Kd_{ianalumab} and Kd_{sBAFF} are respectively the dissociation constants of ianalumab to BAFF-R and of sBAFF to BAFF-R. The above equation assumes a reasonable excess of ligands compared to receptors. It is further assumed that sBAFF concentrations are approximately constant, whereas ianalumab concentrations vary with time.

To determine the BAFF-R occupancy in a fictive disease-related tissue, the following assumptions, supported by in-house experiments, were made:

### Tissue: Serum Concentration Ratio Ianalumab: 1:5

Tissue: Serum concentration ratio sBAFF: 4:1

Table 1 lists all parameters used for tissue receptor occupancy predictions.

TABLE 1
Tissue receptor occupancy model parameters

| Parameter | Definition | Value | | Source |
|---|---|---|---|---|
| Kd_{ianalumab} | Dissociation constant | 0.046 | nM | In vitro experiments |
| | for ianalumab | | | |
| Ka_{sBAFF} | Dissociation constant | 0.73 | nM | In vitro experiments |
| | for sBAFF | | | |
| M_{ianalumab} | Molecular weight of | 150 | kDa | Measured |
| | ianalumab | | | |
| m_{sBAFF} | Molecular weight of | 17 | kDa | Measured |
| | sBAFF | | | |
| CsBAFF in serum | Soluble BAFF | 2.7 | ng/mL | Median at week of |
| | concentration | | | CVAY736X2201 data |
| R_{ianalumab} | Tissue:serum | 1/5 | | AIN457 (skin) |
| | concentration ratio of | | | (Shah and Betts 2013) |
| | ianalumab | | | |
| R_{sBAFF} | Tissue:serum | 4/1 | | “Arbitrary” assumption |
| | concentration ratio of | | | |
| | sBAFF | | | |

Dose Selection Simulations for the Proposed Phase 3 Study in wAIHA

It was assumed that the PK of ianalumab would be comparable in the target wAIHA patient populations as observed in other autoimmune diseases. In addition, based on available literature, baseline B cell in ITP patients is comparable to those previously measured in ianalumab studies in pSS (Toffoletti et al 2008). Given the similarity in pathophysiology between ITP and wAIHA, similar levels of B cells in wAIHA have been used as baseline. Therefore, the final population PK/PD model was used to support the dose and regimen selection in the proposed phase 3 study in wAIHA patients.

In addition, the same hypothesis-driven tissue RO model was also assumed to be appropriate in both autoimmune diseases and wAIHA, therefore the PK-RO model as described in Table 1 was used. Only the level of soluble BAFF concentration in serum was fixed at a lower level of 1.817 ng/mL for wAIHA patients instead of 2.7 ng/ml as in the autoimmune diseases. Of note, serum BAFF concentrations reported in patients with AIHA is 1358.7±141.0 pg/mL (Xu et al 2015), the fixed level at 1.817 ng/ml is chosen for a conservative tissue RO prediction at approximately 3 standard deviation away from the mean.

PKPD simulations were conducted to support selection of the 4-week dose interval using the following criteria:
B cell depletion within the dosing window (<10 cells/μL);
Tissue RO based on a hypothetical model as described in Table 1 (90% threshold);
Ianalumab PK assessed by Ctrough>1.8 μg/mL, which is the steady-state trough concentration for 300 mg q4w s.c., a dose that resulted in the highest clinical response in the multiple-dose, dose-range finding study CVAY736A2201, in pSS patients (week 52 CSR A2201).

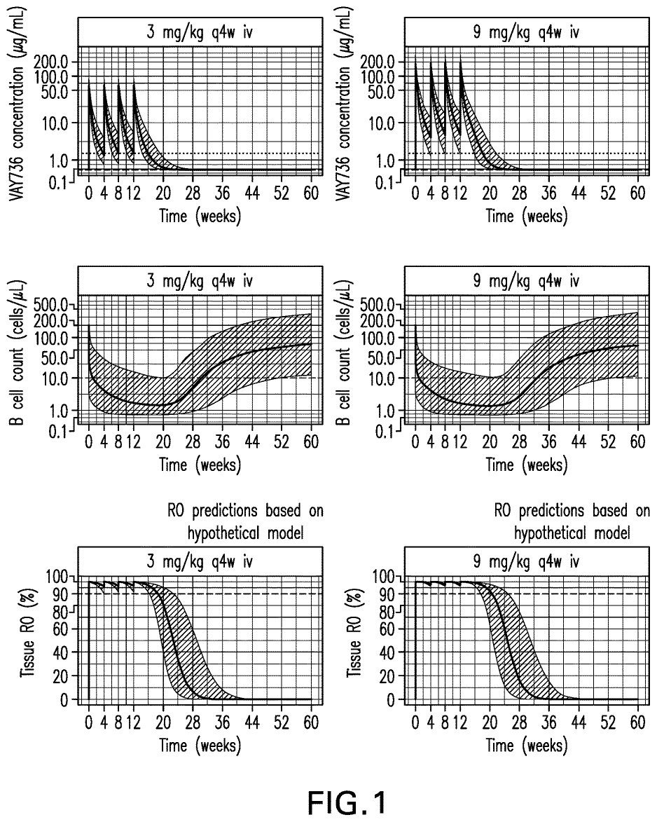

The PKPD simulations FIG. 1 suggest that ianalumab doses and dose regimen at 3 mg/kg and 9 mg/kg, administered every 4 weeks (q4w) i.v., for the treatment period of 16 weeks (4 doses in total) can be considered for the phase 3 study in wAIHA patients. The lower dose at 3 mg/kg iv q4w leads to half of the simulated patients with a trough higher than 1.8 ug/mL, and >95% patients had predicted tissue RO >90% (Table 2). The higher dose at 9 mg/kg i.v. q4w can increase the patients' coverage for PK and tissue RO.

TABLE 2
Characteristics of PK, B-cell and tissue RO for the proposed dosing regimens

| | 3 mg/kg q4w i.v. | | 9 mg/kg q4w i.v. | |
|---|---|---|---|---|
| PK dynamics | | | | |
| % pts with Ctrough >1.8 μg/mL at the last ianalumab | 50% | | 92% | |
| dose | | | | |
| Median (90% PI) of Ctrough (μg/mL) at the last | 1.8 | [0.5-5.9] | 5.5 | [1.5-15.7] |
| ianalumab dose | | | | |
| Median (90% PI) of Cmax (μg/mL) after the last | 62 | [36.7-100] | 182.9 | [110.3-301.2] |
| ianalumab dose | | | | |
| Median (90% PI) of AUC0-28 d (day*μg/mL) after the | 295.8 | [166-540.6] | 880.5 | [470.2-1543.3] |
| last ianalumab dose | | | | |
| Time since last dose for >95% pts with ianalumab | 20 week | | 22 week | |
| concentration <LLOQ | | | | |
| B cell dynamics | | | | |
| Median (90% PI) of total B cell count at the last | 2.1 | [0.4-14.8] | 2 | [0.4-14.9] |
| ianalumab dose | | | | |
| % pts with total B cell count <10 cells/μL at the last | 91% | | 90% | |
| ianalumab dose | | | | |
| Time since last dose for B cell recovery* | 27 week | | 28 week | |
| % pts with B cell recovery* | 61% | | 60% | |
| Median (90% PI) of total B cell count at end of follow- | 68.5 | [11.8-384.8] | 62.9 | [11-412.7] |
| up** | | | | |
| % pts with total B cell count >50 cells/μL at end of | 61% | | 60% | |
| follow-up** | | | | |
| % pts with total B cell count >80 cells/μL at end of | 44% | | 43% | |
| follow-up** | | | | |
| Tissue RO dynamics | | | | |
| Median (90% PI) of predicted RO*** at the last | 97.1 | [90.9-99.1] | 99 | [96.4-99.7] |
| ianalumab dose | | | | |
| % pts with predicted RO*** >90% at the last ianalumab | 96% | | 100% | |
| dose | | | | |

LLOQ = 0.025 μg/mL
*B-cell recovery is defined as ≥80% of baseline or ≥50 cells/μL.
**End of follow-up is defined as 1 year after stopping of ianalumab treatment.
***Predicted tissue RO is calculated based on the trough concentration before the last ianalumab dose.

Mean simulated exposures after the ianalumab 9 mg/kg i.v. dose after last dose (Cmax 192 μg/mL and AUC0-28d 931 day*μg/mL) are within those previously observed at the highest tested i.v. doses, in study in pSS patients after a single i.v. dose of 10 mg/kg (Mean Cmax 213 μg/mL and AUCinf 1140 day*μg/mL, n=12) and study in CLL patients i.v. doses of 9 mg/kg q2w (cycle 3 mean Cmax 262 μg/mL and AUC0-28d,ss 3730 day*μg/mL, 2 doses over 4 weeks, q2w, n=4).

The comparative exposure between the pre-clinical species and mean simulated exposures after ianalumab monthly i.v. doses of 9 mg/kg demonstrates a 14- to 15-fold and 4.3- to 5.6-fold safety margin is observed for Cmax and AUC, respectively, for the monthly 9 mg/kg i.v. dose, indicating the highest ianalumab dose of 9 mg/kg i.v. q4w 9 is safe.

### Example 2: Ianalumab Treatment of wAIHA (Phase III Study)

A clinical study determines the usefulness of ianalumab (VAY736) in the treatment of wAIHA in patients in whom previous treatment failed.

This is a randomized, double-blind, placebo-controlled trial with an open-label cross-over phase to assess the efficacy and safety of ianalumab versuch placebo in warm autoimmune hemolytic anemia (wAIHA) patients who failed previous treatment.

| Objectives | Endpoints |
|---|---|
| Primary | |
| To demonstrate that either dose of ianalumab | Binary variable indicating if a participant achieves |
| induces durable hemoglobin (Hb) response | a durable response (increased Hb) for a period of |
| compared to placebo in patients with wAIHA. | at least 8 consecutive weeks, between W9 and |
| The primary clinical question of interest is: What | W25, in the absence of rescue or prohibited |
| is the effect of either dose of ianalumab versus | treatment |
| placebo (with or without supportive care), in | |
| inducing durable hemoglobin response, in wAIHA | |
| patients ≥18 years of age who failed at least one | |
| line of treatment, regardless of premature | |
| discontinuation from study treatment or temporary | |
| treatment interruption/delay, and in the absence | |
| of rescue or prohibited treatment? | |
| Secondary | |
| Key secondary objective: | Duration of response |
| To demonstrate that either dose of ianalumab | |
| maintains durable hemoglobin response, | |
| compared to placebo | |
| Other secondary objectives: | Time from randomization to achievement of |
| To assess the time to durable | durable response, to first response, to first |
| response/response/complete response in each | complete response |
| treatment group | |
| To assess the quality of response in each | Response rate, complete response rate and |
| treatment group | hemoglobin level |
| To assess the need for rescue treatments in each | Number and proportion of participants that |
| treatment group | receive rescue treatment overall and by type of |
| | rescue treatment |
| | Time-standardized numbers of each type of |
| | rescue treatment |
| | Change from baseline in time-standardized |
| | number of transfusions |
| To assess the safety profile of ianalumab | Frequency of adverse events (AEs) and other |
| | safety parameters |
| To characterize the pharmacokinetics (PK) of | Ianalumab concentration in serum and PK |
| ianalumab | parameters after the first and last dose |
| To assess B-cell levels and immunoglobulin | B-cell levels: |
| levels at each treatment group | Change from baseline in the frequency and |
| | absolute number of CD19+ B cell counts |
| | Time to first occurrence of B cell recovery, |
| | defined as ≥80% of baseline or ≥50 cells/μL |
| | Immunoglobulins: |
| | Change from baseline in immunoglobulin levels |
| To assess the immunogenicity against ianalumab | Incidence and titer of anti-drug antibodies (ADA) |
| | in serum over time |
| To assess the quality of life (QoL) in each | Dedicated Patient Reported Outcomes (PROs) |
| treatment group | |

The treatment of interest consists in i.v. administration (as 2-hour infusion) of ianalumab or placebo every 4 weeks in 4 doses for 4 cycles, with or without the allowed supportive Care. Two different doses level of ianalumab (3 and 9 mg/kg) are administered, compared to placebo, in adult patients with wAIHA who failed previous treatment. The i.v. route of administration is preferred as highest and targeted exposure with established and favorable safety profile in clinical setting across diseases has been achieved using this route. The i.v. route also has the advantage of lower risks to incur into local site reactions, as observed with s.c. dosing, and for which a dose-dependency was observed.

A placebo-controlled, proof-of-concept study CVAY736X2201 conducted in 27 Primary Sjögren Syndrome (pSS) patients provided the first evidence of potential clinical benefit of ianalumab short treatment courses. In this study, an efficient depletion of circulating B cells and clear signs of efficacy, which persisted for a subset of clinical outcomes up to End of Study in the higher dose group, were observed after a single i.v. dose of ianalumab (either 3 mg/kg or 10 mg/kg). Therefore, a 16-week ianalumab treatment duration (4 infusions q4w) was selected for the phase 3 study.

90 participants are randomized in a 1:1:1 ratio to either 3 mg/kg or 9 mg/kg of ianalumab or placebo. Random stratification factor is previous exposure to rituximab prior to randomization (Yes vs No).

Supportive care (which should be stable for at least 4 weeks prior to randomization into the study) can consist of steroids (predniso(lo)ne up to 15 mg/day), erythropoietin alpha, beta, or delta 10 000 units/week, or danazol 200 mg three times/day. Red blood cells transfusions and intravenous immunoglobulins (IVIG) are allowed if the supportive care described above is not sufficient and a participant requires additional support to maintain the safe level of hemoglobin before ianalumab develops its treatment effect.

To avoid potential confounding of efficacy due to effects of transfusions and IVIG (i.v. immunoglobulins), a wash out of 4 weeks has been implemented; only hemoglobin values taken after this wash out period will be used for assessments of efficacy endpoints (durable response, response, complete response).

Prohibited medications are live or live attenuated vaccines, i.v. or oral immunosuppressive drugs, rituximab, belumimab or other monoclonal antibodies, and other experimental therapies.

If clinically indicated (e.g. to ensure patient safety), the treating physician may also administer rescue medication. The investigator should prescribe rescue treatment per local practice, with the exception of the medication listed in the preceding paragraph. The following treatments are considered as rescue medications:
any additional wAIHA-directed treatment which is not listed among prohibited medication
an increase in the dosage of the supportive care >20% comparing to the pre-enrollment dose, which is in line with the recommendations with opinions of the experts. Re-start of allowed supportive treatments at the pre-enrollment dose or return to the pre-enrollment dose level after dose decrease are not considered as a rescue medication.
from Week 8 Day 1 onwards, transfusions is considered a rescue treatment as it could influence the Hb level and confound the efficacy of ianalumab.

Participants eligible for inclusion in this study are male or female patients aged 18 years and older. Patients with primary or secondary wAIHA (previously documented by positive direct antiglobulin test (DAT) specific for anti-IgG or anti-IgA) who had an insufficient response to, or relapsed after at least one line of treatment, including patients with steroid resistance, dependence or intolerance.

Patients' hemoglobin concentration at screening <10 g/dL, associated with presence of symptoms related to anemia (rational: Patients with hemoglobin level below 10 g/dL have decompensated hemolytic anemia and therefore require treatment to increase the level of hemoglobin), and

The dose of supportive care must be stable for at least 4 weeks prior randomization

Participants meeting any of the following criteria are not eligible for inclusion in this study.
Patients with wAIHA secondary to hematologic disease involving bone marrow or other immunologic disease requiring immunosuppressant or presence of other forms of AIHA (cold or intermediate), Evan's Syndrome or other cytopenias.
Prior use of B-cell depleting therapy within 12 weeks prior randomization
Active viral, bacterial or other infections (including tuberculosis or SARS-COV-2)
Known history of primary or secondary immunodeficiency, or a positive human immune deficiency virus (HIV) or positivity for hepatitis C virus (HCV) or hepatitis B virus (HBV).
Live or live-attenuated vaccination within 4 weeks before randomization
Or Patients with following conditions:
   Neutrophils: <1000/mm³
   Serum creatinine >1.5×upper limit of normal (ULN)

Patients with following conditions:
Neutrophils: <1000/mm³
Serum creatinine >1.5×upper limit of normal (ULN)

### Randomized Treatment Period (16 Weeks):

At Week 1 Day 1, patients are randomly assigned in a 1:1:1 ratio to one of the following three arms: Arm A (ianalumab 3 mg/kg intravenously every 4 weeks), Arm B (ianalumab 9 mg/kg intravenously every 4 weeks) or Arm C (placebo intravenously every 4 weeks). During this 16-week randomized treatment phase, participants receive a total of 4 doses of the study treatment, one dose every 4 weeks at the following visits: Week 1 Day 1, Week 5 Day 1, Week 9 Day 1, and Week 13 Day 1. The study treatment is administered via i.v. infusion over 2 hours (+/−15 min). In addition to the study treatment, the patient receives premedication (corticosteroids, paracetamol/acetaminophen or NSAIDs and anti-histamines, as per local institutional guidelines) to mitigate risk of potential systemic infusion-related reactions and supportive care is also allowed in all treatment arms (see preceding paragraph). The randomized treatment phase is the period for assessment of the primary endpoint (see above). After completion of the 16-week randomized treatment period, all participants enter a follow-up period to be monitored for efficacy and safety, or an open-label extension period for placebo treated participants, depending on how the participant responded to the study treatment.
The visit frequency will be every other week (q2w) during the treatment period (week day 1 to week 16) and the primary endpoint follow-up period; for safety monthly during the first 20 weeks from the last dose, then quarterly up to 2 years from the last dose; for efficacy monthly during the first 2 years after the last dose, and then quarterly afterwards until confirmed loss of durable response or end of study

The primary endpoint is a binary variable indicating whether a patient achieves a durable response (Hb ≥10 g/dL and ≥2 g/dL increase from baseline, for at least 8 consecutive weekly assessments) between Week 9 Day 1 and Week 25 Day 1, in the absence of rescue medication or prohibited treatment prior to achieving durable response.

The key secondary efficacy endpoint/variable is: duration of response, defined as
For participants who previously reached durable response: the time from the first hemoglobin assessment showing durable response (as defined in primary endpoint) to confirmed loss of durable response, defined as the first of the following events:
hemoglobin level below 10 g/dL in at least two consecutive weekly assessments* (date of the first of the 2 relapsed assessments will be considered as relapse date),
start of any rescue or prohibited treatment,
death (whatever the cause);
For participants who did not achieve durable response according to the primary endpoint definition: the duration of response will be zero day.

### Example 3 Reduction of Auto-Antibodies in Patients Participating Primary Sjögren's Syndrome (pSS) Trial

In primary Sjogren's syndrome (pSS) trial, 190 patients with pSS were treated with subcutaneous ianalumab (5, 50, 150 or 300 mg q4w for 24 weeks) or placebo (Bowman S J, Fox R, Dorner T, et al (2021) Safety and efficacy of subcutaneous ianalumab (VAY736) in patients with: a randomised, double-blind, placebo-controlled, phase 2b dose-finding trial. Lancet; 399 (10320): 161-171). The study showed a significant dose-response effect of ianalumab on the ESSDAI, and an increase in stimulated salivary flow, with the greatest effect observed at the highest dose.

For quantitative data interpretation for VAY736 effect on autoantibodies in this trial, SSA Ro52kd/TRIM21, SSRo60kd and SSB autoantibodies have been analyzed at same dilution for baseline and post-treatment samples using kit FIDIS™ Connective Profile.

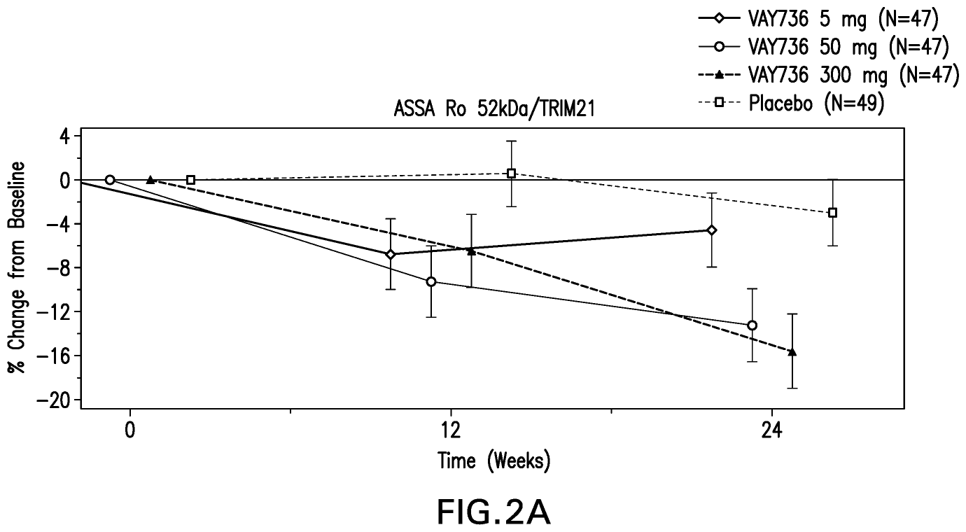

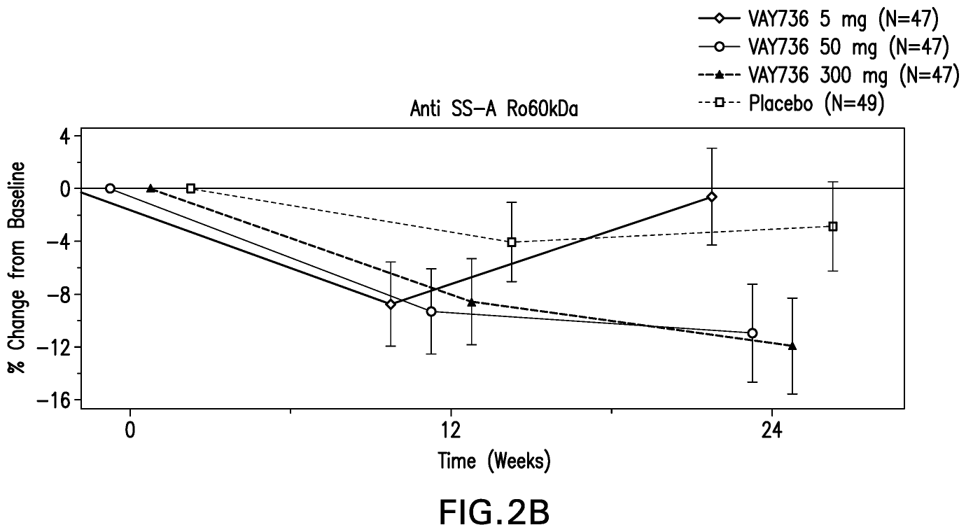

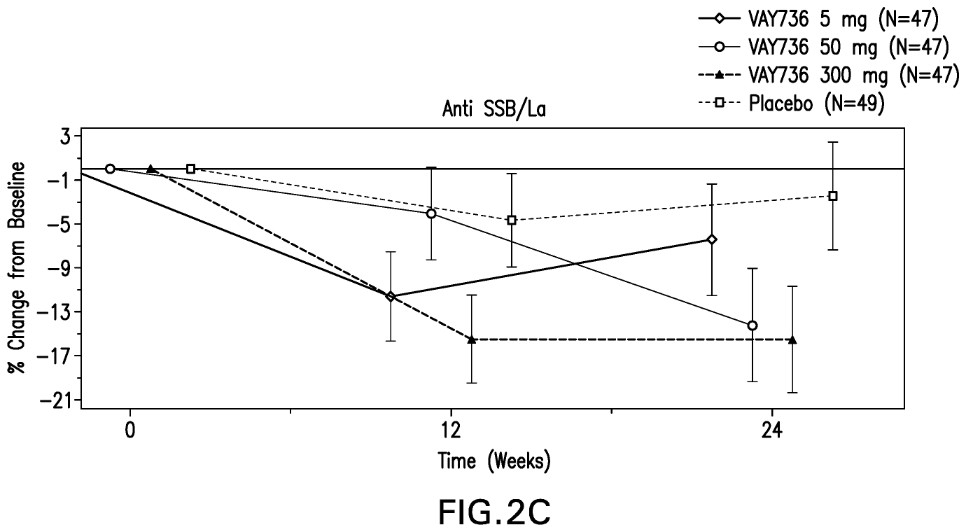

Data below represents data obtained in VAY736 treated arms for SSAro52 (FIG. 2A), SSAro60 (FIG. 2B) and SSB autoantibodies (FIG. 2C) at baseline, week 12 and week 24 using optimal dilution. Data are expressed in % change from baseline. Results clearly shows that decrease of the three autoantibodies upon VAY736 was dose dependent between 5 mg, 50 and 300 mg doses and this decrease of autoantibodies in placebo reached a maximum at week 24 of 4% decrease versus 12 to 16% for 50 and 300 mg VAY736 treated arms.

### Example 4: Reductions of Auto-Antibodies in Patients Participating Systemic Lupus Erythematosus (SLE) Trial

This is a double-blind, randomized, placebo-controlled, multi-center two-arm study, evaluating a dose of 300 mg ianalumab administered s.c. once monthly against placebo, in patients with SLE receiving standard-of-care treatment. The dose regimen for this study is 300 mg s.c. ianalumab once monthly for the treatment period of 18 months.

### Key Inclusion Criteria

Fulfill ≥4 of the 11 American College of Rheumatology 1997 classification criteria for SLE at screening

Patient diagnosed with SLE for at least 6 months prior to screening

Elevated serum titers at screening of ANA (>1:80) in a pattern consistent with an SLE diagnosis, including at a minimum anti-double stranded DNA (anti-ds DNA) or anti-Ro (SSA) or anti-La (SSB) or anti-nuclear ribonucleoprotein (anti-RNP) or anti-Smith (anti-Sm)

Currently receiving corticosteroids and/or anti-malarial and/or thalidomide treatment and/or another DMARD according to the following:

Where corticosteroids are the single standard-of-care medication: an oral dose of ≤30 mg/d for a minimum of 8 weeks prior to randomization and at a stable dose for ≥2 weeks prior to randomization

Where oral corticosteroids are not as a single standard-of-care medication: a stable oral dose of ≤30 mg/d of prednisone or equivalent for a minimum of 8 weeks prior to randomization and at a stable dose for ≥2 weeks prior to randomization

An anti-malarial and/or thalidomide treatment and/or one of the following DMARDs: methotrexate or an imidazole derivative (e.g., azathioprine, mizoribine) or mycophenolic acid derivatives (e.g., mycophenolate mofetil) for a minimum of 12 weeks prior to screening and at a stable dose for ≥8 weeks prior to randomization.

Combination of other DMARDs is not permitted SLEDAI-2K score of ≥6 at screening

BILAG-2004 score at screening of:
At least one “A” in either the mucocutaneous domain or in the musculoskeletal domain,

### OR

one “B” in either the mucocutaneous or musculoskeletal domain AND at least one “A” or “B” in a second domain
Weigh at least 40 kg at screening

### Key Exclusion Criteria

History of receiving prior to screening:
Within 12 weeks: i.v. high dose corticosteroids, calcineurin inhibitors or other oral DMARD except as listed in inclusion criterion 6
Within 24 weeks: cyclophosphamide, or biologics such as intravenous Ig, plasmapheresis, anti-TNF-α mAb, CTLA4-Fc Ig (abatacept) or BAFF targeting agents (e.g., belimumab)
Any B-cell depleting therapies (e.g., anti-CD20 mAb, anti-CD22 mAb, anti-CD52 mAb) or TACI-Ig (atacicept) administered within 52 weeks prior to screening and B-cell count <50 cells/μL at the time of screening

Presence of severe lupus kidney disease as defined by proteinuria above 6 g/day or equivalent using spot urine protein to creatinine ratio, or serum creatinine greater than 2.5 mg/dL (221.05 μmol/L), or requiring immune suppressive induction or maintenance treatment exceeding protocol-defined limits

### Efficacy Assessments:

SRI-4 (SLE responder index)
Physician Global Assessment Visual Analogue Scale (PhGA-VAS)
Patient Global Assessment (VAS)
Flare Rate by BILAG-2004 score
Lupus Low Disease Activity State (LLDAS)

### Results:

The proportion of study patients who achieved the composite primary endpoint at Week 28 of SRI-4 under sustained prednisolone reduction ≤5 mg/d was 42% greater for ianalumab than for placebo. Ianalumab also was better than placebo for the incidence of moderate or severe flares (45% vs 73%, respectively) and time-to-first flare (median not reached vs 11.9 weeks, respectively). At Week 28 the differences between ianalumab and placebo were 50% for proportion of patients achieving SRI-4 response, 34% for reduced corticosteroid use, 43% for the primary combined endpoint of these two outcomes, 20% for Lupus Low Disease Activity State (LLDAS) and 31% for BILAG-based Combined Lupus Assessment (BICLA). Ianalumab was well tolerated without any new safety signals detected during the blinded 28-week treatment period as well as during the open-label treatment period (Week 28 to Week 52) and subsequent safety follow up period.

VAY736 effect on C1q autoantibodies was determined using EnzymeImmunoassay (EIA) provided by BÜHLMANN LABORATORIES, product reference number EK-AC1QA-U.

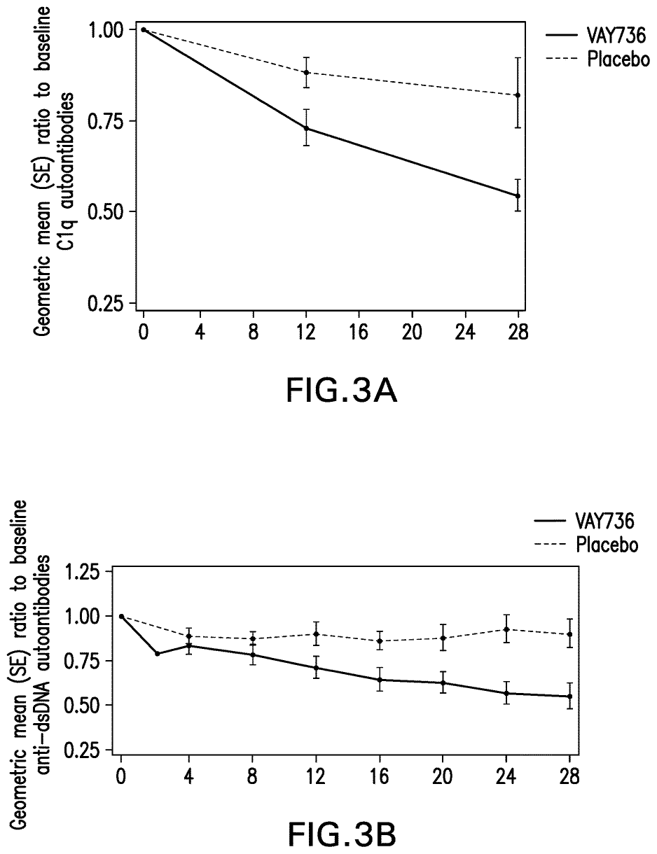

Data below represents data obtained at baseline, week 12 and week24. Data are expressed as geometric mean ratio to baseline. Results clearly shows that decrease of antiC1q autoantibodies upon VAY736 reaching almost 40% versus 18% in the placebo at week 29 ratio to baseline (FIG. 3A).

VAY736 effect on anti-dSDNA autoantibodies was determined with INOVA QUANTA Lite SC ELISA.

Data obtained at baseline, week 12 and week24 are expressed as geometric mean ratio to baseline (FIG. 3B). Results clearly shows that decrease of anti-dSDNA autoantibodies upon VAY736 reaching almost 40% versus 10% in the placebo at week 29 ratio to baseline.

Conclusion: All these data shows that VAY736 decrease autoantibodies in pSS or SLE patients. This effect is VAY736 dose and time dependent. Elevated autoantibodies play important role in the development and progression of AIHA, e.g. wAIHA, which are to be reduced by the treatment with VAY736.

### Example 5 Preclinical Study of the Effects of Ianalumab

The most frequently used murine model of immune thrombocytopenia is a short term passive model that measures the platelet levels following the injection of anti-platelet antibodies, which are inducing platelet elimination through phagocytic cells (Crow A. R et al, Br J Haematology, 2001, 115 (3), 679-86). However, this model is not suitable to study chronic ITP and in particular the role of immune cells in the establishment of the disease. A more recently developed active model of ITP (Chow L. et al. Blood (2010) 115 (6): 1247-1253) presents similarity to the clinical features of human severe ITP and represents more accurately the immune defects of chronic ITP. It has indeed been described to be dependent on the presence of both B cells and T cells to induce a sustained reduction in platelet levels. In this model, CD61-deficient mice are injected with wild-type platelets in order to generated an immune response against anti Splenocytes from the mice are then transferred into severe combined immunodeficiency (SCID) mice, which induces a strong reduction of platelet levels within a week. Both CD19- and CD8-positive populations are required to induce a sustained decreased level of circulating platelets.

Since ianalumab is depleting mouse B cells, we are therefore expecting to see effect on chronic ITP in this mouse model. Splenocytes from mice immune against platelet CD61 are transferred into SCID mice. The mice are treated with 2 to 4 injections of 1) PBS, 2) ianalumab at 10 mg/kg or 3) ianalumab at 100 mg/kg. The mice are bled every week for 4 weeks to evaluate their platelet counts. It is expected to see an increase in platelet counts over time, confirming the efficacy of ianalumab in chronic ITP. Efficacy of ianalumab in this model would confirm its potential in treating B cell mediated cytopenias like ITP, as well as potentially wAIHA.

While various specific embodiments have been illustrated and described, it will be appreciated that various changes can be made without departing from the spirit and scope of the disclosure(s).

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

What is claimed is:

1. An antibody or a binding fragment thereof that inhibits BAFF pathway for use in the treatment of warm autoimmune hemolytic anemia (wAIHA) in a subject.

2. The antibody or a binding fragment thereof of claim 1 being an anti-BAFF antibody or a binding fragment thereof.

3. The antibody or a binding fragment thereof of claim 1 or 2 being an anti-BAFF-R antibody or a binding fragment thereof.

4. The antibody or a binding fragment thereof for use according to any one of the preceding claims, wherein the wAIHA is primary wAIHA.

5. The antibody or a binding fragment thereof for use according to any one of the claims 1-3, wherein the wAIHA is secondary wAIHA.

6. The antibody or a binding fragment thereof for use according to any one of the claims 1-5, wherein the wAIHA is primary or secondary wAIHA and the subject had an insufficient response to, or relapsed after at least one line of treatment, including patients with steroid resistance, dependence or intolerance.

7. The antibody or a binding fragment thereof for use according to any one of the claims 3-6, wherein the antibody or a binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO: 7, and SEQ ID NO:8, respectively.

8. The antibody or a binding fragment thereof for use according to any one of the claims 3-7, wherein the antibody comprises a heave chain having the amino acid sequences of SEQ ID NO: 1 and a light chain having the amino acid sequences of SEQ ID NO:2, respectively.

9. The antibody or a binding fragment thereof for use according to any one of the claims 3-8, wherein the antibody or a binding fragment thereof is ianalumab or a binding fragment thereof.

10. The antibody or a binding fragment thereof for use according to any one of the preceding claims, wherein the antibody or a binding fragment thereof is afucosylated.

11. The antibody or a binding fragment thereof for use according to any one of the preceding claims is belimumab.

12. The antibody or a binding fragment thereof for use according to any one of the preceding claims, wherein the antibody or a binding fragment thereof is to be administered to a subject in need thereof at a dose from about 1 to about 27 mg/kg.

13. The antibody or a binding fragment thereof for use according to any one of the preceding claims, wherein the antibody or a binding fragment thereof is to be administered to a subject in need thereof at a dose of about 1 mg/kg to about 10 mg/kg.

14. The antibody or a binding fragment thereof for use according to claim 12 or 13, wherein the antibody is an anti-BAFF-R antibody.

15. The antibody or a binding fragment thereof for use according to any one of the claims 12 to 14, wherein the antibody is ianalumab.

16. The antibody or a binding fragment thereof for use according to claim 14 or 15, wherein the antibody or binding fragment thereof is to be administered to a subject in need thereof at a dose of about 3 mg/kg.

17. The antibody or a binding fragment thereof for use according to claim 14 or 15, wherein the antibody or binding fragment thereof is to be administered to a subject in need thereof at a dose of about 9 mg/kg.

18. The antibody or a binding fragment thereof for use according to any one of the preceding claims, wherein the antibody or a binding fragment thereof is administered to a subject in need thereof intravenously (i.v.).

19. The antibody or a binding fragment thereof for use according to any one of the preceding claims, wherein the antibody or a binding fragment thereof is to be administered to a subject in need thereof once every 4 weeks (monthly, +/−3 days).

20. The antibody or a binding fragment thereof for use according to any one of the preceding claims, wherein the antibody is ianalumab, wherein ianalumab is to be administered to a subject in need thereof at a dose of 3 mg/kg, intravenously, once every four weeks (monthly, +/−3 days).

21. The antibody or a binding fragment thereof for use according to any one of the preceding claims, wherein the antibody is ianalumab, wherein ianalumab is to be administered to a subject in need thereof at a dose of 9 mg/kg, intravenously, once every four weeks (monthly, +/−3 days).

22. The antibody or a binding fragment thereof that inhibits BAFF pathway for use according to any one of the preceding claims, wherein the subject achieves complete response.

23. The antibody or a binding fragment thereof that inhibits BAFF pathway for use according to any one of the preceding claims, wherein the subject achieves durable response.

24. The antibody or a binding fragment thereof that inhibits BAFF pathway for use according to any one of the preceding claims, wherein the antibody or a binding fragment thereof is to be administered not more than 6 doses or not more than 4 doses.

25. The antibody or a binding fragment thereof that inhibits BAFF pathway for use according to any one of the preceding claims, wherein the antibody or a binding fragment thereof is to be administered at least 4 doses.

26. The antibody or a binding fragment thereof that inhibits BAFF pathway for use according to any one of the preceding claims, wherein the antibody or a binding fragment thereof is to be administered in a total of about 8 doses, about 6 doses, or about 4 doses.

27. The antibody or a binding fragment thereof that inhibits BAFF pathway for use according to any one of the preceding claims, wherein the antibody is ianalumab, wherein ianalumab is is to be administered in a total of 4 doses.

28. The antibody or a binding fragment thereof that inhibits BAFF pathway for use according to any one of the preceding claims, wherein the antibody or a binding fragment thereof is to be administered during a period of up to about 12 months, up to about 9 months, up to about 6 months, up to about 4 months, up to about 3 months, after the first dose administration on day 1, respectively.

29. The antibody or a binding fragment thereof that inhibits BAFF pathway for use according to any one of the preceding claims, wherein the hemoglobin blood concentration of the subject has reached about 10 g/dL or more after administration of the antibody or a binding fragment thereof, wherein preferably the antibody or a binding fragment thereof is ianalumab.

30. The antibody or a binding fragment thereof that inhibits BAFF pathway for use according to any one of the preceding claims, wherein the hemoglobin blood concentration of the subject has increased by about 2 g/dL or more than baseline after administration of the anti-BAFF-R antibody or a binding fragment thereof, wherein preferably the antibody or a binding fragment thereof is ianalumab.

31. The antibody or a binding fragment thereof that inhibits BAFF pathway for use according to any one of the preceding claims, wherein the hemoglobin blood concentration of the subject has reached about 10 g/dL or more and has increased by 2 g/dL or more than baseline after administration of the anti-BAFF-R antibody or a binding fragment thereof, wherein preferably the antibody or a binding fragment thereof is ianalumab.

32. The antibody or a binding fragment thereof that inhibits BAFF pathway for use according to any one of the claims 29 to 31, wherein the increase of the hemoglobin blood concentration takes place after the 3^{rd} dose administration.

33. The antibody or a binding fragment thereof that inhibits BAFF pathway for use according to any one of the claims 29 to 32, wherein the increase of the hemoglobin blood concentration is maintained for at least 3 consecutive weeks, preferably at least 8 consecutive weeks.

34. The antibody or a binding fragment thereof that inhibits BAFF pathway for use according to any one of the claims 29 to 33, wherein the increase of the hemoglobin blood concentration is maintained for at least 3, preferably at least 8 consecutive weeks between about week 9 to about week 25.

35. The antibody or a binding fragment thereof that inhibits BAFF pathway for use according to any one of the preceding claims, wherein the antibody or a binding fragment thereof is administered as monotherapy.

36. The antibody or a binding fragment thereof that inhibits BAFF pathway for use according to any one of the claims 1-34, wherein the antibody or a binding fragment thereof is to be administered in combination with one or more additional agents.

37. The antibody or a binding fragment thereof that inhibits BAFF pathway for use according to claim 36, wherein one or more additional agents are supportive care, wherein preferably the supportive care is selected from a group consisting of corticosteroid, especially prednisone or prednisolone or equivalent, danazole, and erythropoietin (e.g. erythropoietin alpha, beta or delta).

38. The antibody or a binding fragment thereof that inhibits BAFF pathway for use according to claim 36 or 37, wherein the one or more additional agents is a corticosteroid, especially the antibody or a binding fragment thereof that inhibits BAFF pathway is administered when tapering out a corticosteroid.

39. The antibody or a binding fragment thereof that inhibits BAFF pathway for use according to any one of the preceding claims, wherein the subject failed at least one previous line of treatment.

40. The antibody or a binding fragment thereof that inhibits BAFF pathway for use according to claim 39, wherein the previous line of treatment is the same or different wAIHA treatment

41. The antibody or a binding fragment thereof that inhibits BAFF pathway for use according to claim 40, where the different treatment comprised treatment with a corticosteroid, e.g. prednisolone, prednisone or equivalent thereof, or with a B-cell depleting agent, such as rituximab.

42. The antibody or a binding fragment thereof that inhibits BAFF pathway for use according to any one of the preceding claims, wherein the subject has a hemoglobin blood concentration of about 10 g/dL or less, about 9 g/dL or less, about 8 g/dL or less, about 7 g/dL or less, about 6 g/dL or less, or about 5 g/dL or less.

43. The antibody or a binding fragment thereof that inhibits BAFF pathway for use according to any one of the preceding claims, wherein the treatment discontinues after 4 doses, 6 doses, 8 doses, 9 doses or 12 doses.

44. The antibody or a binding fragment thereof that inhibits BAFF pathway for use according to any one of the preceding claims, wherein the treatment discontinues after complete response is achieved.

45. The antibody or a binding fragment thereof that inhibits BAFF pathway for use according to any one of the preceding claims, wherein the treatment discontinues after durable response is achieved.

46. The antibody or a binding fragment thereof that inhibits BAFF pathway for use according to any one of the preceding claims, wherein the subject achieves treatment free remission (TFR).

47. The antibody or a binding fragment thereof that inhibits BAFF pathway for use according to claim 46, wherein the TFR is of a period of about 5 years, 4 years, 3 years, 2 year, 18 months, 16 months, 14 months, 12 months, 9 months, 6 months or 3 months.

48. The antibody or a binding fragment thereof that inhibits BAFF pathway for use according to any one of the preceding claims, wherein the subject has relapsed after TFR, wherein preferably the antibody or a binding fragment thereof that inhibits BAFF pathway is ianalumab; and wherein preferably the subject was administered ianalumab prior to TFR.

49. An antibody or a binding fragment thereof that inhibits BAFF pathway for use according to any one of the preceding claims, wherein the subject is an adolescences 12 to 17 years old.

50. Use of an antibody or a binding fragment thereof that inhibits BAFF pathway in the manufacture of a medicament for the treatment of warm autoimmune hemolytic anemia (wAIHA).

51. A method of treating warm autoimmune hemolytic anemia (wAIHA) in a subject, especially a subject in need thereof, said method comprises administering an antibody or a binding fragment thereof that inhibits BAFF pathway, typically in a therapeutically effective amount.

52. The method according to claim 51, wherein the antibody or a binding fragment thereof is an anti-BAFF antibody or a binding fragment thereof.

53. The method according to claim 51, wherein the antibody or a binding fragment thereof is an anti-BAFF-R antibody or a binding fragment thereof.

54. The method according to any one of the claims 51-53, wherein the wAIHA is primary wAIHA.

55. The method according to any one of the claims 51-53, wherein the wAIHA is secondary wAIHA.

56. The method according to any one of the claims 51-55, wherein the wAIHA is primary or secondary wAIHA, and wherein the subject had an insufficient response to, or relapsed after at least one line of treatment, including patients with steroid resistance, dependence or intolerance.

57. The method according to any one of claims 53-56, wherein the antibody or a binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

58. The method according to any one of the claims 53-57, wherein the anti-BAFF-R antibody comprises a heavy chain having the amino acid sequences of SEQ ID NO: 1 and a light chain having the amino acid sequences of SEQ ID NO:2, respectively.

59. The method according to any one of the claims 53-58, wherein the anti-BAFF-R antibody or a binding fragment thereof is ianalumab or a binding fragment thereof.

60. The method according to any one of the claims 53-59, wherein the antibody or a fragment thereof is an afucosylated antibody.

61. The method according to any one of the claims 52, 54-58 or claim 60, wherein the antibody or a fragment thereof is belimumab.

62. The method according to any one of the claims 51 to 61, wherein the antibody or a binding fragment thereof is administered to a subject in need thereof at a dose from about 1 to about 27 mg/kg.

63. The method according to any one of the claims 51 to 62, wherein the antibody or a binding fragment thereof is administered to a subject in need thereof at a dose of about 1 mg/kg to about 10 mg/kg.

64. The method according to claim 62 or 63, wherein the antibody or binding fragment thereof is an anti-BAFF-R antibody.

65. The method according to any one of the claims 62 to 64, wherein the antibody is ianalumab or a binding fragment thereof.

66. The method according to claim 64 or 65, wherein the antibody or binding fragment thereof is administered to a subject in need thereof at a dose of about 3 mg/kg.

67. The method according to claim 64 or 65, wherein the antibody or binding fragment thereof is administered to a subject in need thereof at a dose of about 9 mg/kg.

68. The method according to any one of claims 51 to 67, wherein the antibody or a binding fragment thereof is administered to a subject in need thereof intravenously (i.v.).

69. The method according to any one of the claims 51 to 68, wherein the antibody or a binding fragment thereof is administered to a subject in need thereof once every 4 weeks (monthly, +/−3 days).

70. The method according to any one of the claims 51 to 69, wherein the antibody is ianalumab, wherein ianalumab is administered to a subject in need thereof at a dose of 3 mg/kg, intravenously, once every four weeks (monthly, +/−3 days).

71. The method according to any one of the claims 51 to 69, wherein the antibody is ianalumab, wherein ianalumab is administered to a subject in need thereof at a dose of 9 mg/kg, intravenously, once every four weeks (monthly, +/−3 days).

72. The method according to any one of the claims 51 to 71, wherein the subject achieves complete response.

73. The method according to any one of the claims 51 to 72, wherein the subject achieves durable response.

74. The method according to any one of the claims 51 to 73, wherein the antibody or a binding fragment thereof is administered not more than 6 doses or not more than 4 doses.

75. The method according to any one of the claims 51 to 74, wherein the antibody or a binding fragment thereof is administered in at least 4 doses.

76. The method according to any one of the claims 51 to 75, wherein the antibody or a binding fragment thereof is administered in a total of about 8 doses, about 6 doses, or about 4 doses.

77. The method according to any one of the claims 51 to 76, wherein the antibody is ianalumab, wherein ianalumab is administered in a total of 4 doses.

78. The method according to any one of the claims 51 to 77, wherein the antibody or a binding fragment thereof is administered during a period of up to about 12 months, up to about 9 months, up to about 6 months, up to about 4 months, up to about 3 months, after the first dose administration on day 1, respectively.

79. The method according to any one of the claims 51 to 78, wherein the hemoglobin blood concentration of the subject has reached about 10 g/dL or more after administration of the antibody or a binding fragment thereof.

80. The method according to any one of the claims 51 to 79, wherein the hemoglobin blood concentration of the subject has increased by about 2 g/dL or more than baseline after administration of the anti-BAFF-R antibody or a binding fragment thereof.

81. The method according to any one of the claims 51 to 80, wherein the hemoglobin blood concentration of the subject has reached about 10 g/dL or more and has increased by 2 g/dL or more than baseline after administration of the anti-BAFF-R antibody or a binding fragment thereof.

82. The method according to any one of the claims 79 to 81, wherein the increase of the hemoglobin blood concentration takes place after the 3^{rd} dose administration.

83. The method according to any one of the claims 79 to 82, wherein the increase of the hemoglobin blood concentration is maintained for at least 3, preferably at least 8 consecutive weeks.

84. The method according to any one of the claims 79 to 83, wherein the increase of the hemoglobin blood concentration is maintained for at least 3, preferably at least 8 consecutive weeks between about week 9 to about week 25.

85. The method according to any one of the claims 51 to 84, wherein the antibody or a binding fragment thereof is administered as monotherapy.

86. The method according to any one of the claims 51-84, wherein the antibody or a binding fragment thereof is administered in combination with one or more additional agents.

87. The method according to claim 86, wherein one or more additional agents are supportive care, wherein preferably the supportive care is selected from a group consisting of corticosteroid, especially prednisone or prednisolone or equivalent, danazole, and erythropoietin (e.g. erythropoietin alpha, beta or delta).

88. The method according to claim 86 or 87, wherein the one or more additional agents is a corticosteroid, especially the antibody or a binding fragment thereof that inhibits BAFF pathway is administered when tapering out a corticosteroid.

89. The method according to any one of the claims 51 to 88, wherein the subject failed at least one previous line of treatment.

90. The method according to claim 89, wherein the previous line of treatment is the same or different wAIHA treatment

91. The method according to claim 90, where the different treatment comprised treatment with a corticosteroid, e.g. prednisolone, prednisone or equivalent thereof, or with a B-cell depleting agent, such as rituximab.

92. The method according to any one of the claims 51 to 91, wherein the subject has a hemoglobin blood concentration of about 10 g/dL or less, about 9 g/dL or less, about 8 g/dL or less, about 7 g/dL or less, about 6 g/dL or less, or about 5 g/dL or less.

93. The method according to any one of the claims 51 to 92, wherein the treatment discontinues after 4 doses, 6 doses, 8 doses, 9 doses, 12 doses.

94. The method according to any one of the claims 51 to 93, wherein the treatment discontinues after complete response is achieved.

95. The method according to any one of the claims 51 to 94, wherein the treatment discontinues after durable response is achieved.

96. The method according to any one of the claims 51 to 95, wherein the subject achieves treatment free remission (TFR).

97. The method according to claim 96, wherein the TFR lasts for a period of about 5 years, 4 years, 3 years, 2 year, 18 months, 16 months, 14 months, 12 months, 9 months, 6 months or 3 months.

98. The method according to any one of the claims 51 to 97, wherein the subject has relapsed after TFR.


SEQUENCE LISTING (18 sequences)

SEQ ID NO: 1 - protein, 454 aa, Homo sapiens
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYDWVPKIGVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Homo sapiens
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 12 aa, Homo sapiens
GDSVSSNSAAWG

SEQ ID NO: 4 - protein, 18 aa, Homo sapiens
RIYYRSKWYNSYAVSVKS

SEQ ID NO: 5 - protein, 12 aa, Homo sapiens
YDWVPKIGVFDS

SEQ ID NO: 6 - protein, 12 aa, Homo sapiens
RASQFISSSYLS

SEQ ID NO: 7 - protein, 7 aa, Homo sapiens
GSSSRAT

SEQ ID NO: 8 - protein, 9 aa, Homo sapiens
QQLYSSPMT

SEQ ID NO: 9 - protein, 453 aa, Homo sapiens
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Homo sapiens
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLGQPKAAPSVTLFPPSSEELQANKATLVCLISDFYPGAVTVAWKADSSPKVAGVETTTPSKQSNNKYAASSYLSLTPEQWKSHRSYSCQVTHEGSTVEKTVAPTECS

SEQ ID NO: 11 - protein, 10 aa, Homo sapiens
GGTFNNNAIN

SEQ ID NO: 12 - protein, 17 aa, Homo sapiens
GIIPMFGTAKYSQNFQG

SEQ ID NO: 13 - protein, 14 aa, Homo sapiens
SRDLLLFPHHALSP

SEQ ID NO: 14 - protein, 7 aa, Homo sapiens
GKNNRPS

SEQ ID NO: 15 - protein, 11 aa, Homo sapiens
QGDSLRSYYAS

SEQ ID NO: 16 - protein, 11 aa, Homo sapiens
SSRDSSGNHWV

SEQ ID NO: 17 - protein, 124 aa, Homo sapiens
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYDWVPKIGVFDSWGQGTLVTVSS

SEQ ID NO: 18 - protein, 110 aa, Homo sapiens
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIKRT